(19) United States
(12) Patent Application Publication
(10) Pub. No.: US 2006/0253185 A1
(43) Pub. Date: Nov. 9, 2006

(54) Catheter for stent delivery having expanded inner member

(75) Inventor: Rudy Beasley, Rohnert Park, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/124,965

(22) Filed: May 9, 2005

(51) Int. Cl. A61F 2/06

(52) U.S. Cl. 623/1.11

(57) ABSTRACT

An inner member for use within a stent delivery system is provided, wherein the stent delivery system has a stent and a balloon disposed within the stent, the balloon configured to be mounted about the inner member in a collapsed state. The inner member comprises a proximal section, a distal section, and an intermediate section. The proximal and distal sections each have a first outer diameter. The proximal and distal sections have a first and a second lumen therethrough, respectively. The intermediate section having a third lumen therethrough in communication with the first and second lumens and has a second outer diameter larger than the first outer diameter for exerting a retaining force on the stent.

### FIELD OF THE INVENTION

The present invention relates generally to an intravascular stent deployment apparatus, and more particularly, to an improved, more flexible stent delivery apparatus capable of being advanced to distal locations.

### BACKGROUND OF THE INVENTION

In a typical percutaneous transluminal coronary angioplasty (PTCA) procedure, a guiding catheter is percutaneously introduced into the cardiovascular system of a patient. The guiding catheter is advanced through a vessel until its distal end is at a desired location in the vasculature. A guidewire and a dilatation catheter having a balloon on the distal end thereof are introduced into the guiding catheter with the guidewire sliding through the dilatation catheter. The guidewire is first advanced out of the guiding catheter into the patient's coronary vasculature. The dilatation catheter is then advanced over the advanced guide wire until the balloon is properly positioned across the lesion. Once in position, the flexible, expandable, preformed balloon is inflated to a predetermined size with a liquid or gas at relatively high pressures (e.g. about ten to twelve atmospheres) to radially compress the artherosclerotic plaque in the lesion against the inside of the artery wall and thereby dilate the lumen of the artery. The balloon is then deflated to a small profile so that the dilatation catheter may be withdrawn from the patient's vasculature and blood flow resumed through the dilated artery.

After angioplasty procedures of the kind described above, there may occur a restenosis of the artery; i.e., a re-narrowing of the treated coronary artery which is related to the development of neointinmal hyperplasia that occurs within the artery after it has been treated via PTCA. In a sense, restenosis is scar tissue that forms in response to mechanical intervention within a vascular structure. To prevent restenosis and strengthen the affected area, an intravascular prosthesis, generally referred to as a stent, can be implanted. The stent is typically delivered to the affected area in a collapsed state via a balloon catheter. The stent is expanded to a larger diameter for placement in the vasculature. This is often accomplished by the balloon portion of the catheter. The stent effectively overcomes the natural tendency of the vessel walls of some patients to close back down, thereby maintaining a normal flow of blood through the vessel that would not be possible if the stent was not in place.

One type of stent which is delivered on a balloon catheter is a generally cylindrical sleeve having a number of openings in its circumference to allow the stent to take on an expanded configuration. In preparation of delivery, the stent is typically collapsed and compressed onto the outside of a collapsed, crimped balloon catheter which may include retainer rings at each end of the stent to help maintain the stent on the balloon. However, the limited securement between the stent and the balloon is not always adequate to insure that the stent will maintain its proper position while being advanced to and through a target lesion. Additionally, the outer surface of the delivery device is uneven because the stent generally extends outwardly beyond the balloon. Thus, the stent may contact a narrow vessel wall and be displaced while the catheter negotiates a narrow vessel and may possibly cause the physician difficulty when attempting to maneuver the stent across the target lesion. In such cases, it may be necessary to pull the stent delivery system back into the guide catheter. In addition, a patient's vasculature may be extremely tortuous to navigate. Because the balloon catheter is relatively stiff across the portion on which the stent is positioned, at times, the stiffness may cause a physician difficulty in negotiating twists and turns in the patient's vasculature.

Thus, a need exists for a stent delivery system that prevents a stent coupled thereto from becoming displaced or dislocated from a predetermined position on the balloon catheter. Moreover, there is a need for a stent delivery system that is flexible so that the ease of navigating a patient's tortuous vasculature is increased. Other desirable features in characteristics of the present invention will become apparent from the subsequent detailed description and the appended claims taken in conjunction with the accompanying drawings.

### BRIEF SUMMARY OF THE INVENTION

According to an aspect of the present invention An inner member configured to be disposed within a stent delivery system is provided, wherein the stent delivery system has a stent and a balloon within the stent, the balloon configured to be mounted about the inner member in a collapsed state. The inner member comprises a proximal section, a distal section, and an intermediate section. The proximal and distal sections each have a first outer diameter, the a first and a second lumen therethrough, respectively. The intermediate section having a third lumen therethrough in communication with the first and second lumens and having a second outer diameter larger than the first outer diameter for exerting a retaining force on the stent.

According to another aspect of the present invention, an inner member is provided that is configured to be disposed within a stent delivery system, wherein the stent delivery system has a stent and a balloon disposed within the stent, the balloon configured to be mounted about the inner member in a collapsed state and having a length in the collapsed state. The inner member comprises a proximal and a distal section, each having a first outer diameter and a first inner diameter, the proximal and distal sections having a first and a second lumen therethrough, respectively. The inner member further comprises an intermediate section having a third lumen therethrough in communication with and bondedly coupled to the first and second lumens, the intermediate section having a second outer diameter larger than the first outer diameter for exerting a retaining force on the stent and a second inner diameter larger than the first inner diameter. The proximal and distal sections comprise a flexible material and the intermediate section comprises material capable of being formed into a desired shape and size and heat treated to maintain said desired shape and size.

### BRIEF DESCRIPTION OF THE DRAWINGS

The following drawings are illustrative of the particular embodiments of the invention and therefore do not limit its scope. They are presented to assist in providing a proper understanding of the invention. The drawings are not to scale and are intended for use in conjunction with the explanations in the following detailed descriptions. The present invention will hereinafter be described in conjunction with the appended drawings, wherein like reference numerals denote like elements, and;

FIG. 1 is a longitudinal view of a stent and balloon assembly in a collapsed state and in accordance with the present invention;

FIG. 2 is a cross-sectional view of the balloon/stent assembly shown in FIG. 1 taken along line 2-2;

FIG. 3 is a longitudinal cross section view of a stent and balloon assembly in an expanded state and in accordance with the present invention; and

FIG. 4 is a longitudinal cross section view of another embodiment of the stent and balloon assembly in an expanded state and in accordance with the present invention.

### DETAILED DESCRIPTION OF THE INVENTION

The following detailed description is merely exemplary in nature and is not intended to limit the scope, applicability, or configuration of the invention in any way. Rather, the following description provides a convenient illustration for implementing an exemplary embodiment of the invention. Various changes to the described embodiment may be made in the function and arrangement of the elements described herein without departing from the scope of the invention. A general description of a stent and balloon assembly will now be provided.

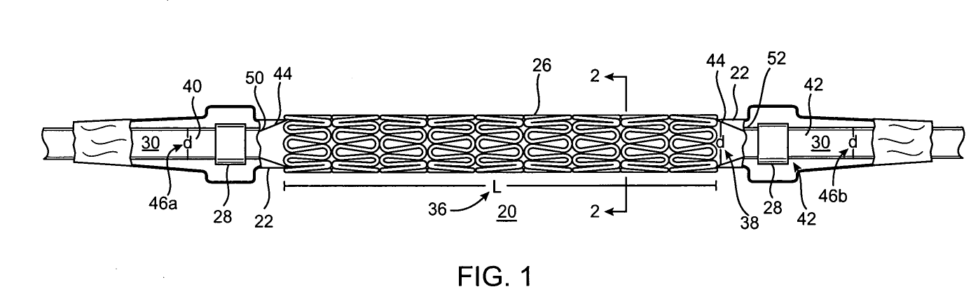

FIG. 1 provides a longitudinal view of a balloon/stent assembly embodying the principles of the present invention. The balloon/stent assembly 20 shown is in a collapsed state and comprises an expandable balloon membrane 22 that is axially mounted around an inner member 24, an expandable stent 26 axially positioned around the expandable balloon 22, and optionally, at least one marker band 28. The inner member 24 comprises a proximal and a distal section 40, 42 coupled to one another by an intermediate section 44. Additionally, a wire lumen 30 extends through the inner member 24. It will be recognized by those skilled in the art that wire lumen 30 is configured for the insertion of a conventional guidewire (not shown) which will enable the balloon/stent assembly 20 to be guided and positioned at a target location in the vessel to be treated.

The expandable balloon 22 is mounted on the inner member 24 in a compressed or collapsed state beneath the stent 26 and extends beyond the proximal and distal ends of the stent 26. The ends of the balloon 22 each sealingly couple and/or adhere to the proximal and distal sections 40, 42 of the inner member 24 to thereby define a space within which the intermediate section 44 portion of the inner member 24 resides. The balloon 22 is generally made of a pliable material such as polyethylene, polyethylene terathalate, PEBAX (polyamide block copolymers and polyester block copolymers), polyvinyl chloride, polyolefin, nylon or the like.

The length of the balloon 22 may be selected to accommodate the particular configuration of the stent to be deployed. When the balloon 22 is collapsed and held beneath the stent 26, the length of the balloon 22 which contacts the stent 26 is a “balloon working length” 36. Additionally, the diameter of the balloon 22 may also be selected depending upon the configuration of the stent. The balloon diameter refers to the inner diameter of the balloon 22 while in a collapsed state beneath the stent 26.

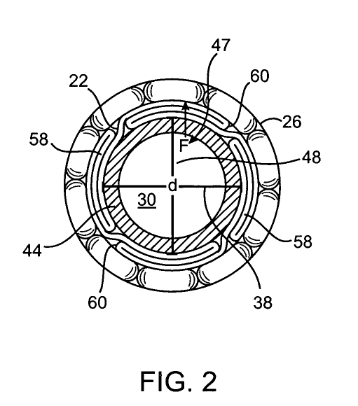

Turning to FIG. 2, a cross-sectional view of one embodiment of the balloon/stent assembly 20 in a collapsed configuration is provided. The cross-section is taken along line 2-2 in FIG. 1. The balloon 22 is collapsed upon the intermediate section 44, producing a plurality of folds (in this case four) 58. Each fold 58 has a longitudinal edge 60. The wings or folds 58 of balloon 22 are formed by pulling the balloon catheter through a forming tool having a generally cylindrical cross-section and defining a terminal opening configured to produce the desired number of wings or folds in the balloon. For example, the terminal opening may include four slits extending radially outward from the end of the forming tool, the number of slits depending upon the number of folds to be produced. As the balloon catheter is pulled through the forming tool, the balloon is pushed through the terminal opening and exits having, for example, four separate flutes. The balloon catheter bearing the fluted balloon portion is then pulled into a sheath, preferably a two-part sheath made of polytetrafluoroethylene or other suitable material so that the flutes fold and wrap around the catheter in a clockwise direction to form a generally spiral configuration. The sheath/balloon catheter assembly is then heated, preferably by placing the assembly in an oven, to form a crease in substantially the length of each of the folded flutes. Following heating, balloon 22 retains the creases formed in the wings to define a generally symmetrical, cylindrical cross-section as can be seen in FIG. 2.

Referring still to FIG. 2, it can be seen that four folds 58 have been formed in balloon 22 each having an edge 60. It should be appreciated, however, that the number of folds 58 might be varied to accommodate different configurations and/or applications. A variety of adhesives is suitable for this purpose; e.g. ultra-violet cure, instant cure, epoxy type, cyanoacrylate type, etc. The stent 26 may then be crimped or compressed onto balloon 22.

Returning to FIG. 1, the inner member 24 is generally tubular and, as briefly mentioned above, comprises proximal and distal sections 40, 42, and an intermediate section 44 coupled therebetween. The proximal and distal sections 40, 42 are, accordingly, generally tubular in shape and are preferably similarly configured, having substantially similar inner and outer diameters. However, as will be appreciated by those with skill in the art, the proximal and distal sections 40, 42 can also be dimensioned so that the proximal section 40 has a smaller inner and/or outer diameter than the distal section 42, or vice versa, depending on any additional feature that may be added or any purpose for which the balloon/stent assembly 20 may be used. The proximal and distal sections 40, 42 are preferably constructed of a flexible, biocompatible material, such as a polyethylene or a polyimide.

The proximal and distal sections 40, 42 each partially define the wire lumen 30. Accordingly, each section 40, 42 has an inner diameter 46a, 46b that is at least slightly greater than the diameter of a guidewire to be inserted therein and advanced therethrough. To aid in the advancement of any guidewire that may be inserted into the lumen 30, the proximal and distal sections 40, 42 may have inner surfaces that are applied or adhered with a lubricious coating. Alternatively, the proximal and distal sections 40, 42 can be constructed of a biocompatible, lubricious material. Examples of such materials include, but are not limited to, varying grades of pebax, or nylon.

The intermediate section 44 comprises first and second ends 50, 52, and a portion of the wire lumen 30 that extends therebetween. The intermediate section 44 is configured to secure the axial position of the stent 26 when it is in a collapsed configuration on the assembly 20. To this end, at least a portion of the intermediate section 44 has an outer diameter 48 that is substantially similar to the balloon diameter 38. FIG. 2 illustrates a cross section of the portion of assembly 20 with the intermediate section 44. Thus, when the stent 26 and the crimped balloon 22 are collapsed around the intermediate section 44, the intermediate section 44 exerts a retaining force 47 against the balloon 22 which is, in turn, exerted on the stent 26 to thereby hold the stent 26 in the desired axial position on the assembly 20.

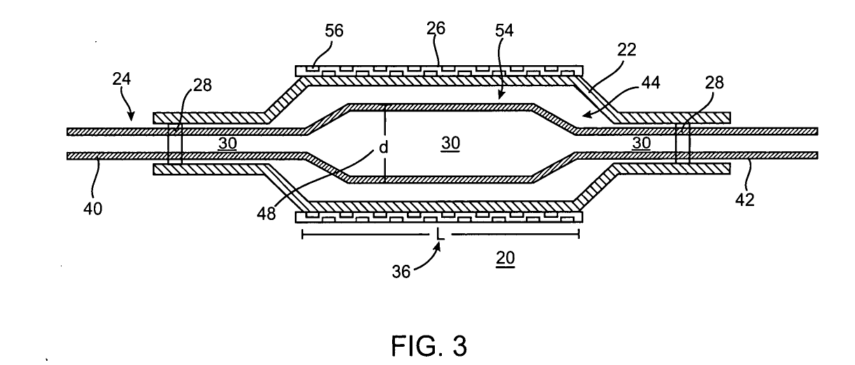

The intermediate section 44 is preferably constructed of a biocompatible, flexible material that permits radial movement so that a physician may mechanically manipulate the intermediate section 44 portion in order to negotiate a patient's tortuous vasculature. As will be appreciated by those with skill in the art, the intermediate section 44 may be constructed of similar or different material as the other portions of the inner member 24. In one embodiment of the present invention, the intermediate section 44 is constructed from material capable of being formed and heat-treated into a desired shape and size. In such an embodiment, the heat-treatment may additionally be employed to alter the modulus of the material. This embodiment is illustrated in FIG. 3. The intermediate section 44 and the proximal and distal sections 40, 42 are integrally formed and/or machined from the same material. However, the portion of the material that comprises the intermediate section 44 is heat treated to maintain a desired configured therein and/or to change the modulus thereof to be sufficiently stiff to maintain the desired shape yet adequately flexible to allow at least some radial movement. Suitable materials include, but are not limited to, thermoplastics, such as polyethylene, pebax, and appropriate metal alloys, such as nickel-titanium alloys.

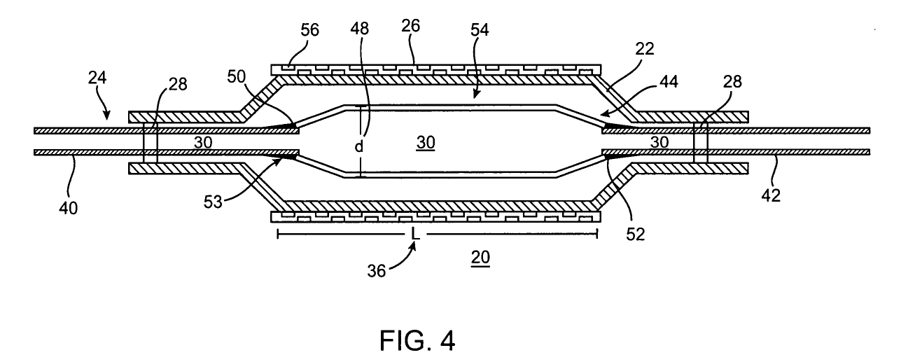

In another embodiment, shown in FIG. 4, the intermediate section 44 is separately constructed from the proximal and distal sections 40, 42 and the expanded member first and second ends 50, 52, are sealingly coupled and/or joined to the proximal and distal sections 40, 42, respectively. This can be achieved by any one of numerous conventional coupling mechanisms or methods. In FIG. 4, the ends 50 52 of the separately constructed intermediate section 44 are bonded 53 to the proximal and distal sections 40, 42. Any one of numerous types of bonding may be employed, such as, for example, ultra-violet cure, instant cure, epoxy type, cyanoacrylate type, etc.

The intermediate section 44 has an outer diameter 48 that is greater than the outer diameters of the proximal and distal sections 40, 42 and, as previously mentioned, is substantially similar to the balloon working diameter 38. The outer diameter 48 of the intermediate section 44 may vary along the length thereof, such as illustrated in FIGS. 3 and 4. Accordingly, the intermediate section 44 may comprise at least a portion of tapering proximate the first and second ends 50, 52 to provide smooth insertion of the assembly 20 when advanced through the vessels. In one embodiment, the size of the outer diameter 48 is substantially uniform in a middle section 54 so as to be level for at least a portion of the intermediate section 44. Alternatively, the size of the outer diameter 48 can become larger until reaching a single point or may increase and decrease along the length of the intermediate section 44, or, can be any other configuration that aids in securing the stent 26 to the assembly 20.

The stent 26 may be constructed of any implantable material having good mechanical strength, such as stainless steel, titanium, tantalum, super-elastic nickel-titanium alloys, or high-strength thermoplastic polymers. The exterior of the stent 26 may be selectively plated with platinum or other implantable radiopaque substance to provide visibility during fluoroscopy. The cross-sectional shape of the finished stent 26 may be circular, ellipsoidal, rectangular, hexagonal, square, or any other desired shape, although a circular or ellipsoidal cross-section is preferable. In one embodiment, the stent 26 comprises a plurality of openings 56 that are configured to allow the stent 26 to collapse and expand. The length and width of the stent 26 is generally determined to a large degree by the size of the vessel into which the stent will be deployed. In a preferred embodiment, the stent 26 is sufficiently dimensioned, for example, length-wise, to maintain its axial orientation without shifting its position on the assembly 20 while exposed to the hydraulics of blood flow. The stent 26 is preferably configured to extend across a significant portion of the target lesion area.

Optionally, marker bands 28, which may be viewed through fluoroscopy, can be included to assist in positioning the assembly.

When the assembly is properly located across a lesion, the balloon 22 may be inflated in a conventional manner. This results in the general uniform, symmetrical expansion of the stent 26 and balloon 22. The amount of inflation and thus the amount of expansion of the stent 26 may be varied as dictated by the lesion itself.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing the exemplary embodiment or exemplary embodiments. It should be understood that various changes can be made in the function and arrangement of elements without departing from the scope of the invention as set forth in the appended claims and the legal equivalents thereof.

What is claimed is:

1. An inner member for use within a stent delivery system, wherein the stent delivery system has a stent and a balloon disposed within the stent, the balloon configured to be mounted about the inner member in a collapsed state, the inner member comprising:
a proximal and a distal section, each having a first outer diameter, the proximal and distal sections having a first and a second lumen therethrough, respectively; and
an intermediate section having a third lumen therethrough in communication with the first and second lumens and having a second outer diameter larger than the first outer diameter for exerting a retaining force on the stent.

2. The inner member of claim 1, wherein the balloon in the collapsed state and the intermediate section each have a length and the balloon length and the intermediate section length are substantially equal.

3. The inner member of claim 1, wherein the proximal and distal sections each have a first inner diameter and the intermediate section has a second inner diameter and the first inner diameter is substantially equal to the second inner diameter.

4. The inner member of claim 1, wherein the proximal and distal sections each have a first inner diameter and the intermediate section has a second inner diameter and the first inner diameter is less than the second inner diameter.

5. The inner member of claim 1, wherein the intermediate section is bondedly coupled to the proximal and distal sections.

6. The inner member of claim 1, wherein the intermediate section is adhesively coupled to said proximal and distal sections.

7. The inner member of claim 1, wherein proximal and distal sections comprise a flexible material.

8. The inner member of claim 1, wherein at least a portion of proximal and distal sections and the intermediate section are integrally formed from a single piece of material.

9. The inner member of claim 8, wherein the intermediate section comprises material capable of being formed into a desired shape and size and heat treated to maintain the desired shape and size.

10. The inner member of claim 1, wherein the second outer diameter is uniform along the length of the intermediate section.

11. The inner member of claim 1, wherein the second outer diameter varies along the length of the intermediate section.

12. The inner member of claim 1, wherein the intermediate section exerts a substantially normal force.

13. An inner member for use within a stent delivery system, wherein the stent delivery system has a stent and a balloon disposed within the stent, the ballon configured to be mounted about the inner member in a collapsed state, the inner member comprising:
a proximal and a distal section, each having a first outer diameter and a first inner diameter, the proximal and distal sections having a first and a second lumen therethrough, respectively; and
an intermediate section having a third lumen therethrough in communication with and bondedly coupled to the first and second lumens, the intermediate section having a second outer diameter larger than the first outer diameter for exerting a retaining force on the stent and a second inner diameter larger than the first inner diameter,
wherein proximal and distal sections comprise a flexible material and the intermediate section comprises material capable of being formed into a desired shape and size and heat treated to maintain said desired shape and size.

14. The system of claim 13, wherein the second outer diameter is uniform along the length of the intermediate section.

15. The system of claim 13, wherein the second outer diameter varies along the length of the intermediate section.

16. The inner member of claim 13, wherein the force exerted by the intermediate section is a substantially normal force.

17. An inner member for use within a stent delivery system, wherein the stent delivery system has a stent and a balloon disposed within the stent, the balloon configured to be mounted about the inner member in a collapsed state and having a length in the collapsed state, the inner member comprising:
a proximal and a distal section, each having a first outer diameter and a first inner diameter, the proximal and distal sections having a first and a second lumen therethrough, respectively; and
an intermediate section having a third lumen therethrough in communication with the first and second lumens, a second outer diameter larger than the first outer diameter for exerting a retaining force on the stent, a second inner diameter that is substantially equal to the first inner diameter, and a length that is substantially equal to the length of the balloon in the collapsed state,
wherein the proximal and distal sections comprise a flexible material and the intermediate section comprises material capable of being formed into a desired shape and size and heat treated to maintain said desired shape and size.

18. The inner member of claim 17, wherein the intermediate section is bondedly coupled to the proximal and distal sections.

19. The inner member of claim 17, wherein the intermediate section is adhesively coupled to said proximal and distal sections.

20. The inner member of claim 17, wherein at least a portion of proximal and distal sections and the intermediate section are integrally formed from a single piece of material.

21. The inner member of claim 17, wherein the second outer diameter is uniform along the length of the intermediate section.

22. The inner member of claim 17, wherein the second outer diameter varies along said length of the intermediate section.

23. The inner member of claim 17, wherein the force exerted by the intermediate section is a substantially normal force.
